(19) United States
(12) Patent Application Publication
(10) Pub. No.: US 2024/0355429 A1
(43) Pub. Date: Oct. 24, 2024

(54) Device and Method for Foodstuff Quality Control

(71) Applicant: Siemens Healthineers AG, Forchheim (DE)

(72) Inventors: David Grodzki, Erlangen (DE); Stephan Biber, Erlangen (DE)

(73) Assignee: Siemens Healthineers AG, Forchheim (DE)

(21) Appl. No.: 18/640,273

(22) Filed: Apr. 19, 2024

(30) Foreign Application Priority Data
Apr. 21, 2023 (DE) 10 2023 203 695.9

(51) Int. Cl. G16C 20/20; G01R 33/20

(52) U.S. Cl. CPC G16C 20/20; G01R 33/20

(57) ABSTRACT

A computer-implemented method for foodstuff quality control may include obtaining, based on a MR measurement, a respective signal strength curve for measurement volume(s) including portion(s) of the sample. The signal strength curve may include a signal strength for measurement time points within a measurement interval, the respective signal strength resulting from an excitation of the measurement volume by an excitation signal radiated during the measurement interval. The method may also include determining a respective ingredient signal curve for the potential ingredients, and determining relative ingredient contents of the potential ingredients. The determination of the ingredient contents may include: minimizing a difference between the signal strength curve for the respective measurement volume and a sum of the ingredient signal curves weighted according to the relative ingredient contents.

### CROSS REFERENCE TO RELATED APPLICATIONS

This patent application claims priority to German Patent Application No. 10 2023 203 695.9, filed Apr. 21, 2023, which is incorporated herein by reference in its entirety.

### BACKGROUND

### Field

The disclosure relates to a system and a computer-implemented method for foodstuff quality control on a sample containing a foodstuff with respect to potential ingredients of the sample.

### Related Art

It may be necessary to test the quality of foodstuffs during production, processing, packaging and, under certain circumstances, also during the storage of foodstuffs. A variety of approaches are known for this purpose, for example chemical analysis of random samples, which, however, require direct access to the actual foodstuff. If, for example, the quality of pre-packaged foodstuffs is to be tested, these methods require the packaging to be opened for selected random samples in order to test their quality.

However, opening packaging in order to test quality is time-consuming, can lead to foodstuff waste and is moreover generally only possible for a small number of random samples at a reasonable cost.

### BRIEF DESCRIPTION OF THE DRAWINGS/FIGURES

The accompanying drawings, which are incorporated herein and form a part of the specification, illustrate the embodiments of the present disclosure and, together with the description, further serve to explain the principles of the embodiments and to enable a person skilled in the pertinent art to make and use the embodiments.

FIG. 1 a device adapted to perform foodstuff quality control according to the disclosure.

FIG. 2 a flow chart of a method for foodstuff quality control according to the disclosure.

The exemplary embodiments of the present disclosure will be described with reference to the accompanying drawings. Elements, features and components that are identical, functionally identical and have the same effect are—insofar as is not stated otherwise—respectively provided with the same reference character.

### DETAILED DESCRIPTION

In the following description, numerous specific details are set forth in order to provide a thorough understanding of the embodiments of the present disclosure. However, it will be apparent to those skilled in the art that the embodiments, including structures, systems, and methods, may be practiced without these specific details. The description and representation herein are the common means used by those experienced or skilled in the art to most effectively convey the substance of their work to others skilled in the art. In other instances, well-known methods, procedures, components, and circuitry have not been described in detail to avoid unnecessarily obscuring embodiments of the disclosure. The connections shown in the figures between functional units or other elements can also be implemented as indirect connections, wherein a connection can be wireless or wired. Functional units can be implemented as hardware, software or a combination of hardware and software.

Thus, the disclosure is based on the object of disclosing an improved approach for foodstuff quality control, which in particular also enables the quality of packaged foodstuffs to be tested without damaging the foodstuff or the packaging thereof.

The object is achieved according to the disclosure by a computer-implemented method for foodstuff quality control on a sample containing a foodstuff with respect to potential ingredients of the sample, comprising the following steps:
obtaining a respective signal strength curve for at least one measurement volume comprising at least one portion of the sample based on a magnetic resonance measurement, wherein the respective signal strength curve describes a respective signal strength for a plurality of measurement time points within a respective measurement interval, which signal strength results from an excitation of the respective measurement volume by an excitation signal radiated at least partially during the measurement interval, and
   ascertaining relative ingredient contents of a plurality of potential ingredients of the sample for the respective measurement volume by specifying a respective ingredient signal curve for the potential ingredients and minimizing the difference between the signal strength curve for the respective measurement volume and a sum of the ingredient signal curves weighted according to the relative ingredient contents.

The method according to the disclosure makes use of the fact that magnetic resonance measurements are carried out in a contactless manner so that the respective signal strength curve used in the method according to the disclosure can also be readily acquired for the respective measurement volume, including for measurement volumes that are, for example, completely or partially within a packaging without opening the packaging. The procedure according to the disclosure is also advantageous for unpackaged foodstuffs since, for example, it is possible to take account of the composition of the foodstuff and thus the quality thereof in regions within the foodstuff without damaging the foodstuff by cutting it open or taking samples.

As a quality criterion, it can, for example, be tested whether certain potential ingredients are not contained or are only contained below specified content limits and/or that the content of unknown substances is small. This will be explained in more detail later.

If the quality criterion is not fulfilled, it is for example possible for further tests to be performed on the sample or parts of the sample, for the sample to be rejected as defective, for the sample to be reprocessed in order to rectify quality defects or for the intended use of the sample to be adapted so that, for example, under some circumstances, depending on their quality, foodstuffs initially intended for human consumption can be used as animal feed instead.

The procedure used according to the disclosure, in which a measured signal strength curve of a magnetic resonance measurement is fitted by a weighted sum of specified ingredient signal curves in order to ascertain properties of a measurement volume, is similar to a procedure used in the field of medical imaging, which is in particular proposed for the classification of brain regions. The use of such a procedure, also known as MR fingerprinting, is, for example, proposed in an article by D. Ma et al., “Magnetic resonance fingerprinting”, Nature 495, 187-192 (2013). An overview of various measurement sequences that can be used for this purpose can, for example, be found in an article by B. B. Mehta et al., “Magnetic resonance fingerprinting: a technical review”, Magn Reson Med. 2019 January; 81 (1): 25-46. In the context of the disclosure, it has been recognized that the approaches for tissue classification in the medical field are surprisingly also very suitable for ascertaining foodstuff quality with respect to potential ingredients of a sample.

Here, it is in principle possible to use any measurement sequences as long as they would result in different potential ingredients, for example due to different T1 and/or T2 times, leading to different signal strength curves if the entire measurement volume were filled by the respective ingredient. This can also be robustly achieved for large quantities of potential ingredients if a sufficiently long measurement interval is used, i.e., if signal strengths are available for at least the same number of, preferably for significantly more, measurement time points than potential ingredients to be distinguished or recognized and repetitions or redundancies in the acquired signal strength curve are avoided, in particular by using a low temporal coherence or, in the case of a spatially resolved measurement, also local coherence of the excitation.

The excitation signal can in particular comprise a plurality of excitation pulses spaced apart in time. The excitation pulses can in particular be separate radiated signals between which the transmission amplitude falls at least approximately to zero. In particular, a plurality of the excitation pulses are radiated during the measurement interval. The sequence of excitation pulses can be selected such that the excitation is spatially and/or temporally incoherent. This at least largely avoids repetitions or very similar portions in the signal strength curve as a result of which even a large number of potential ingredients can be robustly distinguished.

Suitable excitation can, for example, be achieved by pseudorandom variation of the flip angle and/or the phase of the radio-frequency pulse and/or the time interval and/or the echo time in spin-echo measurements and/or the sampling pattern of the K-space in spatially resolved measurements. Additionally, or alternatively, a concatenation of different types of measurement sequences and or/a repetition of sequences with different types of parameterization or the like can be used. For further information on possible measurement sequences, reference is made by way of example to the aforementioned prior art, in particular to the review article by B. B. Mehta et al. Purely by way of example, it is, for example possible to use a sequence of a plurality of “quick-echo splitting NMR” sequences, in particular as flip angles with flip angles that are different from one another. Alternatively, or as part of a sequence of different sequences, it is also possible, for example, to use a “radio-spoiled gradient echo” sequence and/or a “fast imaging with steady-state precision” sequence.

The measurement interval can preferably be longer than 5 seconds and shorter than 2 minutes, in particular between 10 seconds and 1 minute. If spatially resolved acquisition or generally acquisition for a plurality of measurement volumes takes place, the measurement intervals for all measurement volumes or for a subgroup of the measurement volumes can in particular overlap or be identical.

The signal strength curve can in particular be obtained by acquisition by a magnetic resonance device. In particular, the acquired signal strength curve can be further processed immediately or it can initially be stored temporarily. The signal strength curve can in particular be acquired as a step of the method according to the disclosure.

However, the signal strength curve can also be obtained by reception from an external facility, reading it from a memory of a device implementing the method or the like. In particular, it is possible that the acquisition of the signal strength curve is carried out outside the method according to the disclosure and is thus not part of the method according to the disclosure.

The ingredient signal curve can be specified in such a way that, if the respective potential ingredient is present in pure form in the respective measurement volume, apart from potential scaling, it would probably result as a signal strength curve.

The difference between the signal strength curve and the weighted sum of the ingredient signal curves can in particular be minimized by solving an overdetermined system of equations. Here, this results, for each measurement time point, in a weighted sum of the values of the ingredient signal curves for this measurement time point and thus a linear equation for each measurement time point. If the number of measurement time points is greater than the number of potential ingredients considered, this results in an overdetermined linear system of equations. Various approaches for solving overdetermined systems of equations are well known.

For example, a solution can be based on a pseudoinverse matrix. If x is a vector of the initially unknown relative ingredient contents, s is a vector of the signal strength curve with entries corresponding to the signal strengths at the measurement time points, and A is a matrix with columns in each case corresponding to one of the ingredient signal curves, the linear system of equations Ax=b can be achieved by minimizing the 2-norm of the expression Ax−b. If the relative ingredient contents are to be unambiguously determinable, the ingredient signal curves must be linearly independent of one another. In this case, the vector x can be calculated directly according to the equation x=(A^{T}A)⁻¹A^{T}b, which means that the ingredient contents are known.

With suitable calibration of the measuring facility and optimal measuring conditions, the absolute ingredient content can correspond to the relative ingredient content ascertained within the scope of the measuring accuracy. However, in order to minimize errors, for example due to calibration errors and/or environmental influences, absolute ingredient contents can also be calculated on the assumption that, at least approximately, the potential ingredients present in the measurement volume are exclusively those with a known signal curve.

In particular, an absolute ingredient content can be ascertained for at least one of the potential ingredients by dividing the respective relative ingredient content by the sum of all the relative ingredient contents ascertained. This enables the respective ingredient content of the potential ingredients to be scaled in such a way that the sum of the absolute ingredient contents is 1, as a result of which absolute contents of ingredients can be ascertained even in the case of differences in the overall amplitude of the signal strength curve between different samples, for example due to the measurement geometry or environmental influences.

Further treatment of the sample can depend on the fulfilment of a trigger condition, wherein the fulfilment of the trigger condition depends on whether the relative or absolute ingredient content for at least one specific potential ingredient and/or the sum across the relative or absolute ingredient contents across a subgroup of the potential ingredients that does not contain all of the potential ingredients and/or the minimized difference between the signal strength curve and the sum of the ingredient signal curves weighted according to the relative ingredient contents exceed a respective trigger limit value for the measurement volume or at least one of the measurement volumes.

An evaluation of a limit value being exceeded by an ingredient content or the sum of the ingredient contents across the subgroup can, for example, be used to monitor compliance with certain maximum limits for ingredients, for example for specific chemicals or foreign bodies. This can in particular be expedient if different ingredients are to be detected or also if relatively low contents of an ingredient are to be detected within the respective measurement volume. Sums across subgroups can in particular be evaluated if a common trigger limit value is to be used for a plurality of potential ingredients, for example different foreign substances.

The trigger limit value can indicate a permissible upper limit for the content of a potential ingredient or subgroup. However, a sufficiently small trigger limit value enables it to be ensured that the trigger condition is always fulfilled, at least with high probability, if the specific ingredient or an ingredient in the subgroup is present at all. In this case, the trigger limit value corresponds to a tolerance value that is selected as a function of the ascertaining accuracy achieved so that only ingredient contents resulting from expected disturbances or measurement accuracy limits are below the trigger limit. A suitable selection of the trigger limit enables the risks of false-positive triggering and incorrect non-triggering to be weighed up as required.

The acquisition of signal strength curves for a plurality of measurement volumes can, for example, be used to recognize macroscopic inclusions or foreign bodies, for example metal and/or plastic inclusions or the like, in a particularly robust manner. If the measurement volumes are selected as sufficiently small, the properties of such a macroscopic foreign body dominate the acquired signal strength curve. Thus, even if an ingredient signal curve is not available for all of the potential ingredients of possible foreign bodies, the presence of the foreign body can be recognized in a robust manner since this leads to a high residual difference between the signal strength curve and the sum of the ingredient signal curves weighted according to the relative ingredient contents after their minimization.

Generally, the evaluation of the difference between the signal strength curve and the sum of the ingredient signal curves weighted according to the relative ingredient contents after their minimization is advantageous because, apart from noise contributions or measurement inaccuracies, which can be considered by a suitable selection of the trigger limit, this difference is primarily dependent on the presence of unrecognized ingredients. If, for example, ingredient contents are only ascertained for target components of the sample and optionally additionally for specific potentially present foreign substances, if the trigger limit is exceeded by the minimized difference, this is indicative of the presence of at least one other foreign substance in a not insignificant quantity and thus a potential quality problem.

The evaluation of the minimized difference is particularly advantageous for recognizing metallic foreign substances since their magnetic resonance behavior differs greatly from foodstuff components. As a result, the presence of metal particles in the measurement volume can potentially render the relative ingredient contents ascertained inaccurate or falsify them. Since, however, this can be recognized by evaluating the residual difference after minimization, a quality problem caused by a metal inclusion in the foodstuff can still be recognized in a robust manner.

The evaluation of a sum of ingredient contents can be advantageous if different ingredient signal curves result for specific types of ingredients, for example for different muscle tissues or plastics, wherein, however, potentially it is not desired to differentiate between the ingredients of the same type or the subgroup with respect to the quality assessment. In this case, the ingredient contents in this subgroup or this type can be added together and the sum evaluated. In addition to the limit value comparison explained, such a summation can, for example, also be expedient for visualization purposes, for example for a graphical representation of the contents of muscle or fat or the like.

If the trigger condition is fulfilled, the steps of obtaining a respective signal strength curve and of ascertaining relative ingredient contents can in each case be repeated for a plurality of partial volumes of the measurement volume, after which a test is performed for the respective partial volume as to whether a further trigger condition is fulfilled, the fulfillment of which depends on whether the relative or absolute ingredient content for the at least one specific ingredient and/or the sum of the relative or absolute ingredient contents across the subgroup and/or the minimized difference between the signal strength curve and the sum of the ingredient signal curves weighted according to the relative ingredient contents for the respective partial volume exceed the or a further respective trigger limit value.

Thus, the procedure described enables a potential quality problem to be localized. This can, for example, enable the partial volume actually affected to be removed, replaced or specifically re-analyzed. For example, the sample can comprise a carrier, for example a cardboard box or a pallet, that carries a large number of individual foodstuffs or types of foodstuff packaging. Initially, the measurement volume can then comprise a plurality or all of the individual foodstuffs or types of foodstuff packaging. If the trigger condition is fulfilled, i.e., in particular if a foreign body is recognized, for example metal and/or plastic inclusions, decomposition products indicative of the decay of foodstuffs or the like, the individual foodstuffs or types of foodstuff packaging or their content can then be analyzed as separate partial volumes in order to sort out and/or re-analyze problematic foodstuffs or types of foodstuff packaging.

The measurement volume or at least one of the partial volumes can be selected as a function of the spatially resolved magnetic resonance data obtained. For example, a preliminary scan with low spatial resolution, for example with a voxel size of between 1 cm and 5 cm, can be performed in order to localize the at least one relevant measurement volume or the relevant partial volumes containing foodstuffs. The measurement volume or the partial volume can, for example, be localized using contrast based on water content. Thus, it is in particular possible to ascertain the position and/or orientation and/or size of individual foodstuffs or types of foodstuff packaging. Additionally, or alternatively, preliminary information can be used to describe the aforementioned properties. For example, the sample can be positioned in the acquisition region of the magnetic resonance device with an accuracy of a few centimeters or even a few millimeters and the internal structure of the sample, i.e., for example the arrangement of types of foodstuff packaging in a cardboard box or on a pallet, can be known.

In the method explained, in particular a relative ingredient content for water and/or fat and/or muscle tissue and/or pale, soft, exudative (PSE) meat and/or at least one component of spoiled meat can be ascertained. PSE meat is slaughtered pork that is pale, soft and exudative. PSE meat can be recognized immediately after slaughter by its PH value. However, it has been recognized that this meat can also be recognized in a robust manner at later points in time by the explained procedure.

Spoiled meat can in particular be recognized from an analysis of the ingredient contents. Here, for example, degradation products such as amines, hydrogen sulfide, ammonia or toxins can be recognized as separate ingredients, but it is also possible for common compositions of spoiled meat to be directly identified as ingredients and optionally quantified by in each case providing assigned ingredient signal curves for such compositions. Alternatively, to the direct recognition of spoiled meat, its presence can also be generally recognized as a quality defect by evaluating the minimized difference between the signal strength curve and the sum of the ingredient signal curves weighted according to the relative ingredient contents, as explained above.

The ingredient signal curve for at least one of the potential ingredients can be based on a simulation or a calculation based on at least one ingredient parameter of this ingredient. The ingredient curve can in particular be simulated or calculated using the Bloch equation. The simulation of signal intensities based on the Bloch equation is known per se and will therefore not be explained in detail.

The ingredient parameter considered can be the T1 time and/or the T2 time of the respective potential ingredient. These parameters typically have a strong impact on the signal strength curve.

The sample can comprise a plurality of individual foodstuffs or types of foodstuff packaging which are carried by a common carrier, in particular a cardboard box or a pallet. As explained above, in this case, a measurement volume comprising a plurality of or all foodstuffs or types of foodstuff packaging can be analyzed initially. If this identifies a potential contamination or another quality defect, i.e., in particular, if the above-explained trigger condition is fulfilled, smaller partial volumes can then be analyzed, which in particular in each case only comprise one single foodstuff or one single type of foodstuff packaging or preferably their foodstuff-receiving interior.

Obtaining a respective signal strength curve can comprise conveying the sample into an acquisition region of a magnetic resonance device by a conveying facility, in particular a conveyor belt, and the radiation of the excitation signal and the acquisition of the respective signal strength curve for the at least one measurement volume by the magnetic resonance device, wherein the conveying facility automatically guides a plurality of samples into the acquisition region one after the other in order to acquire relative ingredient contents for the respective sample. This enables a large number of samples to be analyzed automatically, wherein this can, for example, take place in a production line and/or packaging line. For example, the samples can be arranged on a conveyor belt in such a way that they are guided into the acquisition region one after the other.

In addition to the method according to the disclosure, the disclosure relates to a system or device with a data processing facility (e.g. processing circuitry, such as one or more processers). The data processing facility may be configured to perform the computer-implemented method according to the disclosure. The data processing facility can, for example, be a programmable data processing facility, which can, in particular, comprise a processor (e.g., a central processing unit (CPU)), a Field-programmable gate array (FPGA), a controller, a microcontroller, or the like, in order to execute a program implementing the method. The data processing facility may generally be referred to as a controller according to the disclosure.

The device can have an input interface via which the signal strength curve can be provided to the device, for example from a magnetic resonance device, from a server or from another data source. Additionally, or alternatively, the device can have an output interface via which, for example, ingredient contents can be provided to an external facility or, for example, visualized via a monitor and/or actuated via the other components in order, for example, to divert samples with a quality defect from a production line. The input interface and the output interface can in each case be hardware interfaces or software interfaces.

The device can, for example, be a workstation computer or a server, be integrated in a magnetic resonance device or form such a device, or also be embodied as a decentralized data processing facility, i.e., implemented as a cloud solution, for example.

The device may include a magnetic resonance device configured to radiate the excitation signal into the at least one measurement volume and to acquire the respective signal strength curve when the sample is arranged at least partially in an acquisition region of the magnetic resonance device. Thus, the device can comprise both data acquisition means and data processing means.

In an exemplary embodiment, the device may include a conveying means, such as a conveyor belt, configured to automatically guide a plurality of samples into the acquisition region one after the other in order to acquire the relative ingredient contents for the respective sample. This enables the quality of a plurality of samples to be tested in a largely automated manner. As mentioned above, this procedure is in particular advantageous if the quality test is to be integrated into a production line or packaging line for foodstuffs, for example as a final control of the foodstuffs.

The disclosure moreover relates to a program for a data processing facility, comprising instructions configured to execute the method according to the disclosure when they are executed on the data processing facility. The program may be embodied on a computer readable storage medium according to the disclosure (e.g. memory).

Features that are described for one of the subjects of the disclosure, i.e., for the computer-implemented method, the system/device, the program or the data carrier can also be transferred to the respective other subject matters of the disclosure with the advantages mentioned.

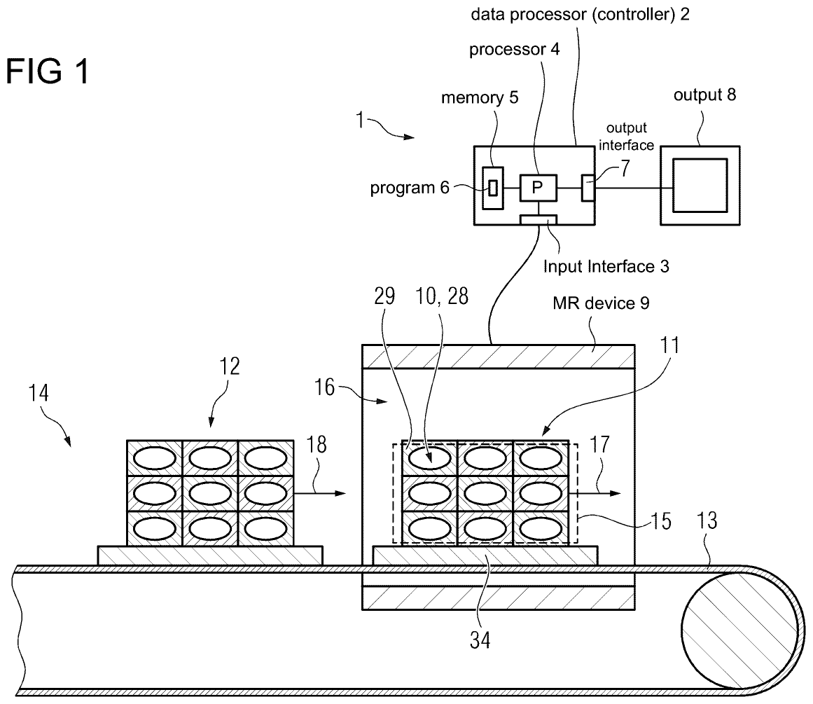

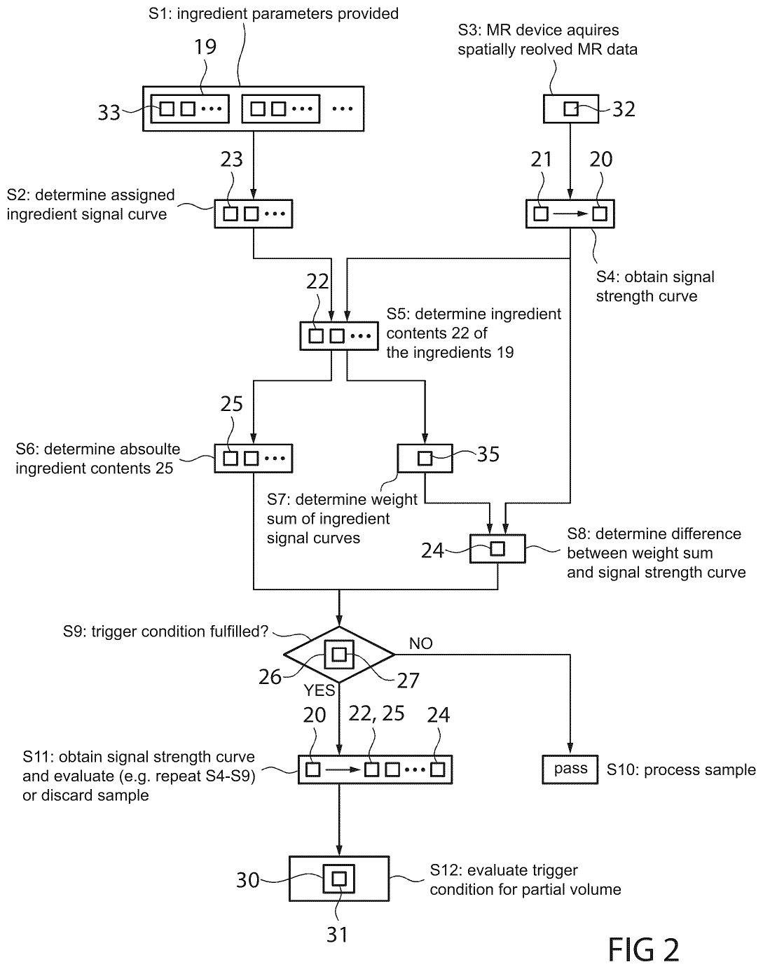

FIG. 1 shows a device 1 which may be adapted to implement a computer-implemented method for foodstuff quality control on a sample 11,12 containing a foodstuff 10 with respect to potential ingredients. The computer-implemented method is described in more detail below with reference to FIG. 2.

In general, the quality control is carried out based on a signal strength curve that is based on a magnetic resonance measurement of the respective sample 11,12. In the example, the signal strength curve is provided to a device 1 via an input interface 3 of a data processing facility (data processor) 2 of the device 1. The data processor 2 may also be referred to as controller 2. In the example, the processing is carried out by a processor 4 which is configured to execute a program 6 that is stored in a memory 5 of the data processor 2. The result of this processing, for example information as to whether the sample satisfies the quality requirements or to what extent undesired ingredients are present, is provided in the example via the output interface 7 to an output device (e.g., a display, speaker, etc.) in order to inform a user about the quality of the respective sample 11,12. The data processor 2 may include processing circuitry that is configured to perform one or more functions/operations of the data processor 2. One or more components (e.g., interfaces 3, 7; processor 4; memory 5) may include processing circuitry configured to perform one or more respective function(s)/operation(s).

In one or more exemplary embodiments, it would also be possible to actuate components of a production line or packaging line 14 in order to influence the further processing of the sample 11,12 in order, for example, to send samples 11, 12 with quality defects for separate processing or a follow-up control via a switch.

In the example, the device 1 may comprise a magnetic resonance (MR) facility (“MR device”) 9 configured to radiate an excitation signal into the measurement volume 15 and to acquire the respective signal strength curve when the respective sample 11,12 is arranged in the acquisition region 16 of the magnetic resonance device 9. Moreover, the device 1 may comprise a conveyor belt 13 adapted to guide a plurality of samples 11,12 sequentially into the acquisition region 16 in order to perform the quality control.

When the quality control has been completed, the conveying means (conveyor, conveyor belt or the like) 13 of the device 1 can be actuated in order to send the sample 11, as indicated by the arrow 17, out of the acquisition region 16 for further processing and, at the same time, to guide the next sample 12, as indicated by the arrow 18, into the acquisition region 16 of the magnetic resonance facility 9. The quality control for the sample 12 can then be performed.

A possible implementation of the method for foodstuff quality control is explained in the following with additional reference to FIG. 2. Here, the central point is that, in step S4, a signal strength curve 20 is obtained for at least one measurement volume 15, wherein the signal strength curve 20 is based on a magnetic resonance measurement. The signal strength curve 20 comprises a respective signal strength for a plurality of measurement time points within a respective measurement interval, which signal strength results from an excitation of the respective measurement volume 15 by an excitation signal 21 radiated at least partially during the measurement time interval.

In step S5, then in each case a relative ingredient content 22 is ascertained for a plurality of potential ingredients 19 by minimizing a difference 24 between the signal strength curve 20 for the respective measurement volume 15 and a sum 35 of specified ingredient signal curves 23 weighted according to the relative ingredient contents 22. The minimization can be carried out using conventional optimization methods by varying the relative ingredient contents 22. However, as already mentioned in the general part, the optimum ingredient contents 22 can typically be calculated directly from the ingredient signal curves 23 and the signal strength curve 20 without having to explicitly calculate the weighted sum 35 or the difference 24.

For better understanding of these central steps and to illustrate possible further advantageous embodiments of the method, in the exemplary embodiment shown in FIG. 2, these steps are embedded in a more comprehensive method for foodstuff quality control.

In the exemplary embodiment, in step S1, ingredient parameters 33 are initially specified for the potential ingredients 19 to be considered, in particular the T1 and T2 times of the potential ingredients 19.

In step S2, an assigned ingredient signal curve 23 is then ascertained by simulation based on the Bloch equation for each of these potential ingredients 19. Here, the ingredient signal curves 23 can be ascertained by assuming in the simulation that in each case only the assigned potential ingredient 19 is present in the entire measurement volume.

In order to select a suitable measurement volume 15 for the acquisition of the signal strength curve 20 or to find suitable partial volumes 28 for later measurements, in step S3, the magnetic resonance facility 9 can optionally acquire spatially resolved magnetic resonance data 32. Here, it is possible to use a relatively coarse resolution with voxel sizes of, for example, a few centimeters. The selection of a suitable contrast, for example, the water content, the position and/or orientation of the sample 11 with respect to the magnetic resonance facility 9 and/or its dimensions can be determined in order to be able to select a suitable measuring range 15. Partial volumes 28 can then, for example, be selected such that they in each case only comprise the foodstuff 10 within one of the types of foodstuff packaging 29. If sufficiently accurate positioning of the sample 11 has already been achieved with respect to the measuring facility 9 by means of the conveying facility 13 or with the aid of further sensors and if the internal structure of the sample 11 is known, step S3 can be omitted.

In step S4, the signal strength curve 20 is obtained. Here, the signal strength curve 20 could in principle be read from a data source. However, in the example, it is acquired directly with the aid of the magnetic resonance facility 9.

As already discussed in the general part, here, the excitation signal 21 is preferably selected such that a temporal coherence between the excitations and, in the case of spatially resolved measurements, also local coherence are avoided as far as possible in order to be able to obtain as much information as possible about relevant ingredient parameters 33 or their impact on the signal strength curve 20 for a given length of measurement interval. Possible measurement sequences have also already been discussed in the general part.

In step S5, as explained above, the relative ingredient contents 22 of the ingredients 19 to be considered can then be ascertained. In principle, it may be sufficient to ascertain these relative ingredient contents 22 for foodstuff quality control. For example, it may already be recognized in this step that a relative ingredient content that is beyond a limit value is ascertained for an undesired potential ingredient 19. The limit value can, for example, correspond to the maximum relative ingredient content that is likely to result from noise or other expected disturbances. Thus, based on the ascertaining of relative ingredient contents 22, samples 11,12 containing an undesired ingredient can be recognized if its ingredient signal curve 23 is known.

In the context of quality control, it can also be desirable to test whether potential ingredients 19 that are per se permissible or even desirable are present in excess. For example, when testing the quality of meat, a certain fat content can be permissible or even desirable. However, if the fat content exceeds a certain limit, this may constitute a quality defect.

In principle, to recognize a quality defect of this kind, it is possible to compare the relevant relative ingredient content 22 with a limit value. However, since the overall amplitude of the acquired signal strength curve 20 can be dependent on numerous influencing factors, for such applications, it is generally advantageous initially to ascertain absolute ingredient contents 25 in step S6.

Here, it can be assumed that the respective sample 11,12 consists, at least to a large extent, of the potential ingredients 19 considered and other components are only present in traces. As will be explained later, if this is not the case, this can also be recognized and assessed as a quality defect. Thus, the absolute ingredient content 25 can be calculated or estimated in a very good approximation by dividing the relative ingredient content 22 of the ingredient 19 for which the respective absolute ingredient content 25 is to be ascertained by the sum of all relative ingredient contents 22 ascertained.

In the exemplary embodiment discussed, it should also be considered to be a quality defect if the signal strength curve 20 indicates the presence of ingredients for which no considered ingredient signal curve is present. In order to achieve this, in step S7, a sum 35 of the ingredient signal curves 23 weighted according to the relative ingredient contents 22 ascertained in step S5 is ascertained. For this purpose, the ingredient signal curves 23 can in each case be multiplied by the relative ingredient content 22 of the corresponding ingredient 19 and then added together.

In step S8, a difference 24 between this weighted sum 35 and the signal strength curve 20 is then ascertained. This can, for example, be carried out by subtracting the weighted sum 35 from the signal strength curve 20 and then ascertaining a measure for the resulting difference signal, for example a sum of the squares of the difference value for the different measurement time points.

In step S9, it is then tested whether a trigger condition 26 is fulfilled. For this purpose, in the example, the absolute ingredient contents 25 for specific, in particular undesired, potential ingredients 19 and the difference 24 are in each case compared with a trigger limit value 27. If one of the trigger limit values 27 is exceeded, the trigger condition 26 is fulfilled, since either undesirable or unknown ingredients are present to a not insignificant extent and this is interpreted as a quality defect in the example.

If the trigger condition 26 is not fulfilled in step S9, in step S10, the respective sample 11,12 can then be further treated as normal, since no quality defect has been recognized. For example, the sample 11,12 can be guided to the end of a production line or packaging line and transported away from there.

On the other hand, if the trigger condition 26 is fulfilled in step S9, the sample 11,12 is then further treated in another way in order to respond to the apparent quality defect. In the simplest case, the respective sample 11,12 could be separated out and discarded.

However, to avoid foodstuff waste, in the described exemplary embodiment, initially a more precise localization of the potential quality defect is performed. Specifically, it is necessary to ascertain which foodstuff 10 or which foodstuff within which foodstuff packaging 29 has a potential quality defect so that targeted follow-up analysis of this foodstuff 10 or these types of foodstuff packaging 29 can be carried out or the defective types of foodstuff packaging 29 can be removed from the sample 11,12 and replaced by defect-free types of foodstuff packaging 29.

This is achieved by repeating in step S11 the obtaining of the signal strength curve 20 and its evaluation, i.e., in particular the ascertaining of the relative or absolute ingredient contents 20,25 and the difference 24 for a plurality of partial volumes 28 of the measurement volume 15. Apart from the different acquisition region of the measurements, this can be carried out as described above for steps S4 to S8.

As already explained above, the partial volumes can in particular correspond to the regions in which the foodstuff 10 is located in the individual types of foodstuff packaging 29 so that quality control is carried out selectively for the individual foodstuff 10 or the individual types of foodstuff packaging 29 in order to recognize defective foodstuffs 10 or types of foodstuff packaging 29.

Specifically, for this purpose, in step S12, a further trigger condition 30 for the respective partial volume 28 can be evaluated; this fulfilled if a respective further trigger limit value 31 is exceeded by the absolute ingredient content 25 of a respective specific ingredient and/or difference 24 ascertained for the respective partial volume 28.

As explained above, foodstuffs 10 or types of foodstuff packaging 29, for which the further trigger condition 30 is fulfilled can then be removed from the sample 11 or from the pallet serving as a carrying means 34 and in particular replaced by defect-free foodstuffs 10 or types of foodstuff packaging 29.

In the example shown in FIG. 1, this can be carried out by issuing a corresponding message on the display facility 8 (e.g., by providing an electronic signal to generate a visual and/or audible notification). Additionally, or alternatively, the evaluation may automate corresponding processes by actuating further components of the device 1 (e.g., by generating a corresponding control signal to cause a respective action), for example gripper arms, transport switches, conveyor belts and the like, as a function of the fulfilment of the trigger condition 26 and/or the respective further trigger condition 30.

Although the disclosure has been illustrated and described in more detail by the preferred exemplary embodiment, the disclosure is not limited by the disclosed examples and other variations can be derived herefrom by the person skilled in the art without departing from the scope of protection of the disclosure.

To enable those skilled in the art to better understand the solution of the present disclosure, the technical solution in the embodiments of the present disclosure is described clearly and completely below in conjunction with the drawings in the embodiments of the present disclosure. Obviously, the embodiments described are only some, not all, of the embodiments of the present disclosure. All other embodiments obtained by those skilled in the art on the basis of the embodiments in the present disclosure without any creative effort should fall within the scope of protection of the present disclosure.

It should be noted that the terms “first”, “second”, etc. in the description, claims and abovementioned drawings of the present disclosure are used to distinguish between similar objects, but not necessarily used to describe a specific order or sequence. It should be understood that data used in this way can be interchanged as appropriate so that the embodiments of the present disclosure described here can be implemented in an order other than those shown or described here. In addition, the terms “comprise” and “have” and any variants thereof are intended to cover non-exclusive inclusion. For example, a process, method, system, product or equipment comprising a series of steps or modules or units is not necessarily limited to those steps or modules or units which are clearly listed, but may comprise other steps or modules or units which are not clearly listed or are intrinsic to such processes, methods, products or equipment.

References in the specification to “one embodiment,” “an embodiment,” “an exemplary embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

The exemplary embodiments described herein are provided for illustrative purposes, and are not limiting. Other exemplary embodiments are possible, and modifications may be made to the exemplary embodiments. Therefore, the specification is not meant to limit the disclosure. Rather, the scope of the disclosure is defined only in accordance with the following claims and their equivalents.

Embodiments may be implemented in hardware (e.g., circuits), firmware, software, or any combination thereof. Embodiments may also be implemented as instructions stored on a machine-readable medium, which may be read and executed by one or more processors. A machine-readable medium may include any mechanism for storing or transmitting information in a form readable by a machine (e.g., a computer). For example, a machine-readable medium may include read only memory (ROM); random access memory (RAM); magnetic disk storage media; optical storage media; flash memory devices; electrical, optical, acoustical or other forms of propagated signals (e.g., carrier waves, infrared signals, digital signals, etc.), and others. Further, firmware, software, routines, instructions may be described herein as performing certain actions. However, it should be appreciated that such descriptions are merely for convenience and that such actions in fact results from computing devices, processors, controllers, or other devices executing the firmware, software, routines, instructions, etc. Further, any of the implementation variations may be carried out by a general-purpose computer.

The various components described herein may be referred to as “modules,” “units,” or “devices.” Such components may be implemented via any suitable combination of hardware and/or software components as applicable and/or known to achieve their intended respective functionality. This may include mechanical and/or electrical components, processors, processing circuitry, or other suitable hardware components, in addition to or instead of those discussed herein. Such components may be configured to operate independently, or configured to execute instructions or computer programs that are stored on a suitable computer-readable medium. Regardless of the particular implementation, such modules, units, or devices, as applicable and relevant, may alternatively be referred to herein as “circuitry,” “controllers,” “processors,” or “processing circuitry,” or alternatively as noted herein.

For the purposes of this discussion, the term “processing circuitry” shall be understood to be circuit(s) or processor(s), or a combination thereof. A circuit includes an analog circuit, a digital circuit, data processing circuit, other structural electronic hardware, or a combination thereof. A processor includes a microprocessor, a digital signal processor (DSP), central processor (CPU), application-specific instruction set processor (ASIP), graphics and/or image processor, multi-core processor, or other hardware processor. The processor may be “hard-coded” with instructions to perform corresponding function(s) according to aspects described herein. Alternatively, the processor may access an internal and/or external memory to retrieve instructions stored in the memory, which when executed by the processor, perform the corresponding function(s) associated with the processor, and/or one or more functions and/or operations related to the operation of a component having the processor included therein.

In one or more of the exemplary embodiments described herein, the memory is any well-known volatile and/or non-volatile memory, including, for example, read-only memory (ROM), random access memory (RAM), flash memory, a magnetic storage media, an optical disc, erasable programmable read only memory (EPROM), and programmable read only memory (PROM). The memory can be non-removable, removable, or a combination of both.

1. A computer-implemented method for foodstuff quality control on a sample containing a foodstuff with respect to potential ingredients of the sample, the method comprising:
obtaining, based on a magnetic resonance measurement, a respective signal strength curve for at least one measurement volume comprising at least one portion of the sample, wherein the respective signal strength curve includes a respective signal strength for a plurality of measurement time points within a respective measurement interval, the respective signal strength resulting from an excitation of the respective measurement volume by an excitation signal radiated at least partially during the measurement interval;
determining a respective ingredient signal curve for a plurality of the potential ingredients of the sample;
determining relative ingredient contents of the plurality of the potential ingredients of the sample for the respective measurement volume, wherein the determining the relative ingredient contents includes: minimizing a difference between the signal strength curve for the respective measurement volume and a sum of the ingredient signal curves weighted according to the relative ingredient contents; and
providing, based on the determined relative ingredient contents, an electronic signal to facilitate further processing of the sample.

2. The computer-implemented method as claimed in claim 1, further comprising determining an absolute ingredient content for at least one of the potential ingredients based on the respective relative ingredient content and a sum of all ascertained relative ingredient contents.

3. The computer-implemented method as claimed in claim 2, wherein determining the absolute ingredient content comprises dividing the respective relative ingredient content by the sum of all ascertained relative ingredient contents.

4. The computer-implemented method as claimed in claim 2, wherein further treatment of the sample depends on fulfilment of a trigger condition, the fulfilment of the trigger condition being based on whether:
the relative or absolute ingredient content for at least one specific potential ingredient exceeds a respective trigger limit value for the measurement volume or at least one of the measurement volumes; and/or
the sum across the relative or absolute ingredient contents across a subgroup of the potential ingredients that does not contain all of the potential ingredients exceeds a respective trigger limit value for the measurement volume or at least one of the measurement volumes;
and/or the minimized difference between the signal strength curve and the sum of the ingredient signal curves weighted according to the relative ingredient contents exceeds a respective trigger limit value for the measurement volume or at least one of the measurement volumes.

5. The computer-implemented method as claimed in claim 4, wherein, in response to the trigger condition being fulfilled, the method comprising:
repeating the obtaining the respective signal strength curve and the determining the relative ingredient contents for a plurality of partial volumes of the measurement volume; and
determining, for a respective partial volume of the plurality of partial volumes, whether a further trigger condition is fulfilled, the fulfillment of which depends on whether:
the relative or absolute ingredient content for the at least one specified ingredient exceeds the respective trigger limit value or a further respective trigger limit value; and/or
the sum of the relative or absolute ingredient contents across the subgroup exceeds the respective trigger limit value or the further respective trigger limit value; and/or
the minimized difference between the signal strength curve and the sum of the ingredient signal curves weighted according to the relative ingredient contents for the respective partial volume exceeds the respective trigger limit value or the further respective trigger limit value.

6. The computer-implemented method as claimed in claim 4, wherein the measurement volume or at least one of the partial volumes is selected as a function of obtained spatially resolved magnetic resonance data.

7. The computer-implemented method as claimed in claim 1, wherein the measurement volume is selected as a function of obtained spatially resolved magnetic resonance data.

8. The computer-implemented method as claimed in claim 1, further comprising determining a relative ingredient content for: water; fat; muscle tissue; pale, soft, exudative (PSE) meat; and/or at least one component of spoiled meat.

9. The computer-implemented method as claimed in claim 1, wherein determining the respective ingredient signal curves for at least one of the plurality of potential ingredients is based on: a simulation; or a calculation based on at least one ingredient parameter of the at least one of the plurality of potential ingredients.

10. The computer-implemented method as claimed in claim 9, wherein the at least one ingredient parameter considered is a T1 time and/or a T2 time of the respective at least one of the plurality of potential ingredients.

11. The computer-implemented method as claimed in claim 1, wherein the sample comprises a plurality of individual foodstuffs or types of foodstuff packaging carriable by a common carrier.

12. The computer-implemented method as claimed in claim 1, wherein obtaining a respective signal strength curve comprises:
conveying the sample into an acquisition region of a magnetic resonance device by a conveyor; and
radiating the excitation signal and acquiring the respective signal strength curve for the at least one measurement volume by the magnetic resonance device,
wherein the conveyor is configured to automatically and consecutively guide a plurality of samples into the acquisition region to acquire relative ingredient contents for the respective plurality of samples.

13. The computer-implemented method as claimed in claim 1, wherein the electronic signal is configured to generate a notification corresponding to the determined relative ingredient contents.

14. The computer-implemented method as claimed in claim 1, wherein the electronic signal is a control signal configured to control a device to process the sample.

15. A non-transitory computer-readable storage medium with an executable program stored thereon, that when executed, instructs a processor to perform the method of claim 1.

16. A device adapted for foodstuff quality control on a sample containing a foodstuff with respect to potential ingredients of the sample, the device comprising:
one or more processors; and
memory storing instructions that, when executed by the one or more processors, cause the device to:
obtain, based on a magnetic resonance measurement, a respective signal strength curve for at least one measurement volume comprising at least one portion of the sample, wherein the respective signal strength curve includes a respective signal strength for a plurality of measurement time points within a respective measurement interval, the respective signal strength resulting from an excitation of the respective measurement volume by an excitation signal radiated at least partially during the measurement interval;
determine a respective ingredient signal curve for a plurality of the potential ingredients of the sample;
determine relative ingredient contents of the plurality of the potential ingredients of the sample for the respective measurement volume, wherein the determining the relative ingredient contents includes: minimizing a difference between the signal strength curve for the respective measurement volume and a sum of the ingredient signal curves weighted according to the relative ingredient contents; and
provide, based on the determined relative ingredient contents, an electronic signal to facilitate further processing of the sample.

17. The device as claimed in claim 16, further comprising a magnetic resonance device that is configured to: radiate the excitation signal into the at least one measurement volume, and acquire the respective signal strength curve when the sample is arranged at least partially in an acquisition region of the magnetic resonance device.

18. The device as claimed in claim 17, further comprising a conveyor configured to automatically and consecutively guide a plurality of samples into the acquisition region to acquire the relative ingredient contents for the respective plurality of samples.
